(19) United States
(12) Patent Application Publication
(10) Pub. No.: US 2010/0233089 A1
(43) Pub. Date: Sep. 16, 2010

(54) IMAGING OF GENETIC MATERIAL WITH MAGNETIC RESONANCE

(75) Inventors: Brian D. Ross, Altadena, CA (US); Pratip Bhattacharya, Pasadena, CA (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: HUNTINGTON MEDICAL RESEARCH INSTITUTES, Pasadena, CA (US)

(21) Appl. No.: 12/680,733

(22) PCT Filed: Oct. 6, 2008
(86) PCT No.: PCT/US08/78999

§ 371 Date: May 28, 2010

Related U.S. Application Data
(60) Provisional application No. 60/978,091, filed on Oct. 5, 2007.

(30) Foreign Application Priority Data
Oct. 5, 2007 (US) 60978091

(51) Int. Cl. A61K 49/12; A61P 35/00

(52) U.S. Cl. 424/9.3

(57) ABSTRACT

A method for imaging genetic material such as DNA, RNA and genes by magnetic resonance imaging incorporating hyperpolarization techniques, such as PASADENA or DNP may be used in various app metabolomics, medical diagnosis and genetic research.

### FIELD OF SUBJECT MATTER

The present subject matter relates to methods for imaging of genetic material using magnetic resonance. Specifically, the present subject matter relates to methods for imaging of DNA, RNA and genes using magnetic resonance imaging techniques incorporating hyperpolarization.

### BACKGROUND OF THE SUBJECT MATTER

All publications herein are incorporated by reference to the same extent as if each individual publication or patent application was specifically and individually indicated to be incorporated by reference. The following description includes information that may be useful in understanding the present subject matter. It is not an admission that any of the information provided herein is prior art or relevant to the presently claimed subject matter, or that any publication specifically or implicitly referenced is prior art.

Magnetic resonance (“MR”) imaging has become a well-accepted and commonly-used technique for studying a wide range of physiologic processes. The present subject matter is useful in connection with disease diagnosis and prognosis, and in the broader study of biological systems. Indeed, many hospitals and medical facilities have MR imaging equipment on-site, and routinely make use of it to aid in the diagnosis and monitoring of an array of diseases and physiologic conditions. MR imaging has been used to study the products of gene expression, but imaging of polynucleotides and genes have not previously been possible. Along these lines, there remains a strong need for MR imaging techniques that allow for the direct imaging of DNA, RNA and genes.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 is a graph depicting the typical reproducibility achieved on a series of three experiments conducted within 30 minutes of one another on ¹³C spectrum of 3.5 mM 1-¹³C-succinate-d2 hyperpolarized at pH=2.9, yielding 17.6±1.4% polarization.

FIG. 2 is a graph depicting the ¹³C spectrum of 3.5 mM 1-¹³C-succinate-d2 hyperpolarized at pH=2.9, using naturally abundant ethanol with 207 mM of ¹³C per carbon site as a reference. Spectra were acquired at 4.7T with a Bruker Avance console and home-built double resonant probe.

FIG. 3 is an image depicting the hyperpolarized succinate image of a rat brain.

FIG. 4 depict the gial and neuronal cycles for adding glutamine and glutamate C1 and C5 carbons enriched with ¹³C label from C1 of succinate in tumors.

FIG. 5 is an Ex vivo ¹³C high resolution NMR spectra at 11.7T of succinate metabolism from brain and tumor tissues, collected one hour after a mixture of 16 mM 1-¹³C-maleate and 8 mM 1-¹³C-succinate was injected into the carotid artery of a 9L tumor-bearing rat. The order of magnitude contrast ratio between tumor and brain suggests that hyperpolarized 1-¹³C-succinate is a highly potent molecular imaging agent.

FIG. 6 is an image demonstrating access to structural and dynamic elements of nucleotides through hyperpolarization.

FIG. 7 illustrates that single scan hyperpolarized ³¹P NMR spectrum of a seven base-pair of fully-matched palindromic DNA duplex (5′-GACAGTC-3′) reveals seven distinct phosphorus resonances from the fourteen backbone phosphates because of the presence of C2 axis of symmetry due to self-complementary nature of the sequence (FIG. 7A), while single scan hyperpolarized ³¹P NMR spectra of a seven base-pair of single-mis-matched (AA) palindromic DNA duplex (5′-GACAGTC-3′) reveals many more, well resolved phosphorus resonances than were seen in FIG. 7A signal (>7) from the fourteen backbone phosphates (FIG. 7B).

### DETAILED DESCRIPTION OF THE SUBJECT MATTER

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); March, Advanced Organic Chemistry Reactions, Mechanisms and Structure 4th ed., J. Wiley & Sons (New York, N.Y. 1992); and Sambrook and Russell, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods or materials similar or equivalent to those described herein, which could be used in the practice of the present subject matter. Indeed, the present subject matter is in no way limited to the methods or materials described. For purposes of the present subject matter, the following terms are defined below.

“Dynamic Nuclear Polarization” or DNP is a solid state polarization technique using unpaired electrons to reach a spin order of unity within hours.

“Hyperpolarization” includes any MR technique which employs spin physics and/or chemistry to elevate the nuclear alignment of a material substantially above that induced by the prevailing magnetic field (Boltzmann polarization).

“Metabolites” are the intermediates and products of metabolism. The term metabolite is usually restricted to small molecules. A primary metabolite is directly involved in the normal growth, development, and reproduction of cells. A secondary metabolite is not directly involved in those processes, but usually has important ecological function, such as antibiotics and pigments.

“Metabolomics” is the systematic study of the unique chemical fingerprints that specific cellular processes leave behind; specifically, the study of their small-molecule metabolite profiles.

“Metabonomics” is defined as the quantitative measurement of the dynamic multiparametric metabolic response of living systems to pathophysiological stimuli or genetic modification.

“PASADENA” is an acronym for Parahydrogen and Synthesis Allows Dramatically Enhanced Nuclear Alignment, and is a chemical method of reaching spin-order of unity within seconds at liquid state temperature using chemical synthesis.

Metabolites are the products of gene expression and while magnetic resonance imaging (“MRI”) cannot image a metabolite, magnetic resonance spectroscopy (“MRS”) can do so. Conventional MR therefore offers an indirect approach to imaging the live gene, through their metabolic products or absences. Starting with McArdle's syndrome, a hereditary muscle disorder caused by absence of glycogen phosphorylase, (Ross B. D., Radda G. K., Gadian D. G., Rocker G., Esiri M. Falconer-Smith J. Examination of a case of suspected McArdle's syndrome by 31P nuclear magnetic resonance. N Engl J. Med.; 204(22):1338-1342 [1981]), phosphofructokinase deficiency (Chance B. et al., ³¹P NMR studies of control of mitochondrial function in phosphofructokinase-deficient human skeletal muscle. Proc Natl Acad Sci USA.; 79(24):7714-7718 [1982]) and mitochondrial encephalomyopathy (electron transport succinic dehydrogenase, site II deficiency) (Radda et al., A mitochondrial encephalomyopathy. A combined 31P magnetic resonance and biochemical investigation. J. Neurol. Sci. 1985 November; 71(1):105-18 [1982]) it has been clear that non-invasive biological and medical identification of gene deletions can be achieved with conventional NMR. Over the succeeding years, a similar body of information has been acquired from the brain. A variety of hereditary leukodystrophies, Canavan disease, vanishing white matter disease, hepatic enzyme defects (Ornithine transcarbamylase deficiency), MELAS, chromosomal abnormalities (e.g. Down's syndrome) and the hereditary dementias (e.g. Alzheimer's Disease) (Danielson et al., Magnetic Resonance Spectroscopy Diagnosis of Neurological Diseases, Marcel Dekker, [New. York, N.Y.: 1999]) have all yielded indirect images of a ‘live’ gene in the human brain by way of the gene metabolic products.

Because of the rich information content of metabolites available in an NMR spectrum, be it proton (1H), carbon (13C) or phosphorus (31P), the science of NMR metabolomics has emerged (Lindon, J. C. et al., The Handbook of Metabonomics and Metabolomics, Elsevier Science & Technology Books [2007]). When applied to urine, blood or cerebrospinal fluid, as many as 500 individual metabolites might be identified in vitro for data mining and pattern recognition and contribute significantly to in vitro imaging of gene expression. Even though MRS in vivo is markedly less well resolved, using a number of available techniques, the number of metabolites identified may reach 100 (Ross et al., 2003).

Enrichment of metabolic pools with stable (non-radioactive) isotopes 13C and 15N allows MR determination of flux through amino acids, glycolysis, the TCA cycle and the urea cycle. Applications in the brain extend to determination of neuronal-glial cycling and quantification of neurotransmitter rates in animals (Cerdan S. Role of Glial Metabolism in Diabetic Encephalopathy as Detected by High Resolution ¹³C NMR. NMR Biomed 16: 440-449 [2003]) and humans, in healthy individuals and in a wide variety of neurological and psychiatric diseases.

A certain subset of genes, the oncogenes, are believed to represent a potent cause of cancer, and imaging the metabolite products of their expression using MRS has been another much explored application of in vivo NMR. Starting in the early 1980's with human skin tumors (Griffiths et al., Application of MRS in Cancer in Pre-clinical Models. Springer Netherlands, [1981]), human hypernephroma, and Wilms kidney tumor, perfused ex vivo, abnormal metabolites of the choline-phosphocholine series appeared to characterize a malignant tumor in comparison to adjacent benign tissue. Therapeutic interventions directed at the malignant tissue impacted the choline-metabolite series within minutes, offering a non-invasive method for tumor monitoring (Lancet et al., Monitoring Response to Chemotherapy of Intact Human Tumours by 31P Nuclear Magnetic Resonance. [1984]). Examples include brain tumor, prostate cancer, breast cancer and lymphomas. (B-Cell lymphoma of bone responded within days to effective chemotherapy by alterations in the choline metabolite series). Although the gene substrate is not fully understood, the excess of choline metabolites in untreated tumors is universal, and in vivo proton MRS has been widely used to diagnose tumors and to record their response to therapy.

The MAP-kinase cascade involves an intra-cellular osmolyte, myoinositol (“ml”) (Haussinger, D. et al., Activation of extracellular signalregulated kinases ERKI and ERK2 and the c-Jun N-terminal kinase JNK2 by hyperosmotic stress in H41 IE rat hepatoma cells: Cross-talk by induction of MAP kinase phosphatase (MKP1) expression and role in development osmoprotection. Eur. J. Cell Biol. 72:80. [1997]), which can in turn be quantified in proton MRS and natural abundance 13C MRS. Observation of elevated ml in low grade malignant brain tumors suggests that proton MRS is in fact imaging ml, the product of the oncogene, MAP-kinase.

Using MR, it is possible to image a number of live genes in the human brain through their metabolites. A narrow range of oncogenes may have incidentally been ‘imaged’ in this way, but the number is rather small. Of the 100,000 genes believed to control brain function, we are indirectly looking at only 10 to 20 and only those rather arbitrarily made visible through very high concentrations, typically in the mM range, of their expressed metabolic product. To date, because of its low sensitivity, MR has not permitted direct imaging of live genes. That task has been addressed instead by positron emission tomography (“PET”), which has several orders of magnitude greater sensitivity (10⁻¹°, versus 10⁻³ for conventional MRI).

Briefly, many of the techniques of molecular biology have been directly applied to in vivo imaging with PET (Tavitian, B. et al., In vivo imaging of oligonucleotides with positron emission tomography. Nature Medecine, 4:467-471, [1998]). This has been achieved through radioactive labeling of anti-sense oligonucleotides, attaching probes for cell transport and blood brain barrier permeability and conjugation to a monoclonal antibody (Pardridge, W. M. et al., Drug Targeting of a Peptide Radiopharmaceutical through the Primate Blood-Brain Barrier in vivo with a Monoclonal Antibody to the Human Insulin Receptor., J. Clin. Invest. [1997]).

Because, as suggested above, MR has far greater specificity than many other non-invasive techniques, including PET, several advances in MR technology are currently being explored to advance methods of MR imaging. MR may also be preferred over PET because MR does not employ radioactive isotopes. MR also has pursued the molecular biology path with some success. Gd-based smart contrast agents which enhance MR signal contrast (without increasing MR signal, however) and iron (Mion) tagging of targeted probes remain of great interest. In the quest for greater sensitivity, high field MR (2-5 fold signal gain) has been advocated.

However, among the most important advances in MR of recent years, and the one most likely to produce advances in direct gene imaging, is hyperpolarization.

Hyperpolarized MR signal enhancements of 10,000 to 100,000 fold have been achieved in conventional MRI scanners. With this advance, MR sensitivity approaches that of PET. Hyperpolarization may be achieved through any MR technique which employs spin physics and/or chemistry to elevate the nuclear alignment substantially above that induced by the prevailing magnetic field (Boltzmann polarization). Table 1 lists several methods for hyperpolarization by nonequilibrium phenomena.

TABLE 1
Various methods for hyperpolarization by nonequilibrium phenomena

| | Hyperpolarization Methods |
|---|---|
| 1 | ONP near optically spin-polarized electrons in atoms, |
| | semiconductors, organic triplets. |
| 2 | CIDNP in geminate pair recombination products |
| 3 | Dynamic Nuclear Polarization (DNP) near unpaired |
| | electrons |
| 4 | Cross-relaxation to high polarizable species (e.g. ¹²⁹Xe) |
| 5 | ‘Brute Force’ by high magnetic field and low temperature |
| 6 | Parahydrogen and Synthesis Allows Dramatically |
| | Enhanced Nuclear Alignment (PASADENA) in products |
| | of molecular addition of parahydrogen |

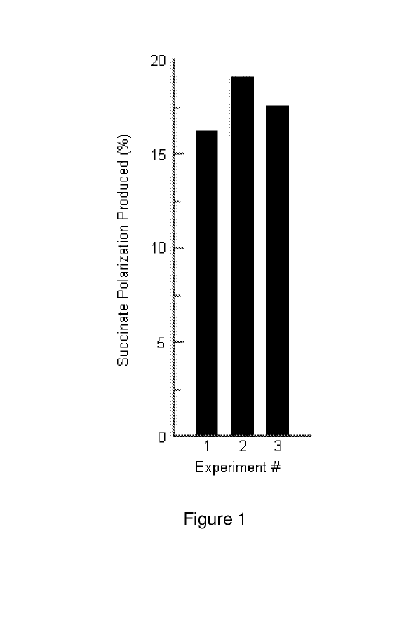

The degree of hyperpolariation realized in practice is important. Experiments have shown that PASADENA achieves highly reproducible hyperpolarization 18%+1-1% (N=15) (see FIG. 1). Estimates in the literature vary between 0.5% (PHIP, Bargon), 10% (DNP, Chen) and 60% (Xe; Hersman).

Hyperpolarized signal decays very rapidly, with a T1 of only tens of seconds. Several approaches to speeding ¹³C MR acquisition take advantage of the large signal produced by hyperpolarization, as well as the most prevalent disadvantage, its very short T1.

The greatest advances are occurring in the design of molecular imaging agents for PASADENA and DNP. Of biological relevance are 13C pyruvic acid, lactate, acetate and glutamine, for which there are proof of concept demonstrations with DNP. Maleate, succinate, a vascular-plaque binding reagent, TFPP and choline have shown efficient hyperpolarization and early promise with PASADENA.

PASADENA is a chemical method of reaching spin-order of unity within seconds at liquid state temperature using chemical synthesis. Dynamic Nuclear Polarization (“DNP”) is a solid state polarization technique using unpaired electrons to reach a spin order of unity within hours. Both of the techniques are capable of rendering over 10,000 fold signal enhancement which overcomes previous sensitivity limitations of in vivo NMR spectroscopy.

In the PASADENA method of hyperpolarization, parahydrogen is used for creating highly polarized nuclei, replacing the thermal equilibrium polarization determined by the Boltzmann Distribution by polarizations of order unity in a growing variety of molecular species. The parahydrogen gas is used in a chemical reaction (hydrogenation by catalytic molecular addition to the unsaturated bond of a PASADENA precursor) to produce the PASADENA product of interest. In order to preserve the spin correlation between the protons immediately after hydrogenation, a rhodium catalyst is used which transfers the protons as a unit on to the precursor, without scrambling. The PASADENA phenomenon was invented in 1986 by Bowers and Weitekamp (Bowers, C. R. and Weitekamp, D. P. Transformation of Symmetrization Order to Nuclear-Spin Magnetization by Chemical-Reaction and Nuclear-Magnetic-Resonance. Phys. Rev. Lett., 57(21): p. 2645-2648 [1986]; Bowers, C. R. and Weitekamp, D. P. Parahydrogen And Synthesis Allow Dramatically Enhanced Nuclear Alignment. J. Am. Chem. Soc., 109(18): p. 5541-5542 [1987]), and creates a non-equilibrium spin order that can be transformed into polarization. The first biochemical application of the technique was reported in 2001 (Golman, K. et al., Molecular imaging with endogenous substances. Proc Natl. Acad. Sci. USA. 100(18): p. 10435-9 [2003]). The transfer of this spin order into polarization of a suitable hetero nucleus can be accomplished by either a diabatic field-cycling scheme or by RF pulses (Golman, K. et al., Real-time Metabolic Imaging. Proc. Natl. Acad. Sci., 103: p. 11270-11275 [2006]), before administration of the hyperpolarized agent to the experimental subjects. The chemistry and the spin order transfer takes place at an elevated temperature (40-60° C.) within the hydrogenation reactor. Then the hyperpolarized substance is filtered to remove the rhodium catalyst before NMR experiments, stopping the hydrogenation and removing the toxic component.

The DNP methodology (Golman, K. et al., Molecular imaging with endogenous substances. Proc Natl. Acad. Sci. USA. 100(18): p. 10435-9 [2003]) uses low temperature, high magnetic field, and the unpaired electron of selected species (e.g., triaryl radical) to strongly polarize nuclear spins in the solid state. The solid sample is subsequently dissolved rapidly in water to create a solution of molecules with hyperpolarized nuclear spins. The polarization is performed in a DNP polarizer, consisting of a separate superconducting magnet (3.35T) and a liquid-helium cooled sample space. The DNP process entails irradiating the frozen sample with 94 GHz microwaves. Subsequent to polarization, the sample is dissolved by an injection system inside the DNP magnet and the toxic triaryl radical removed by a membrane filter. The dissolution process effectively preserves the nuclear polarization.

It is now well established that NMR signal is enhanced over 10,000 fold by the PASADENA and DNP methodologies of creating nuclear spin polarization with recent improvements (Golman, K. et al., Real-time Metabolic Imaging. Proc. Natl. Acad. Sci., 103: p. 11270-11275 [2006]; Bhattacharya, P. et al. Ultra-fast three dimensional imaging of hyperpolarized ¹³C in vivo. MAGMA, 18(6): 245-56 [2005]; Bhattacharya, P. et al., Towards Hyperpolarized ¹³C Succinate Imaging of Brain Cancer. Journal of Magnetic Resonance, 186: p. 108-113 [2007]). Regardless of the particular pulse sequence or detection method, the sensitivity is proportional to the fractional polarization P of the target spins, for example, P=1×10⁻⁶ for ¹³C at equilibrium at 1.5 T and ambient temperature. It is well known that P for a given nucleus is conserved through chemical reactions, relaxing toward the equilibrium value with a characteristic time T₁ of up to several tens of seconds for ¹³C. Thus, the establishment by any such method of a high value of P allows the corresponding sensitivity enhancement to be transported to any location and chemical species that can be reached on this time scale. Recent work has demonstrated ¹³C polarizations in excess of 20% (P>0.2) for the nascent products of molecular addition of dihydrogen and DNP and sub-second imaging of these products following arterial injection. This polarization decays with time constant equal to the familiar spin-lattice relaxation time T₁ but even after 5 T₁ (from 1 to 6 minutes for the molecules proposed) the available signal is still more than 2000 times greater than the equilibrium ¹³C signal. Thus, there is time for the hyperpolarized molecules to be delivered via the blood flow, taken up into extracellular and intracellular volumes, and even metabolized before data acquisition. The signal-to-noise ratio at 5 T₁ with hyperpolarization may be achieved with ordinary polarization only after more than 50 days of signal averaging at 1 s⁻¹.

Accordingly, in vivo imaging of live genes is feasible. By enhancing MR signal 10,000 to 100,000 times and demonstrating feasibility for in vivo imaging of metabolic events, including some which are reflective of oncogene activity, hyperpolarized MR will challenge PET in the imaging of a live genes and other nucleotides.

Therefore, in accordance with one embodiment of the present invention, genetic material in the form of, for example, a live gene, DNA, RNA, an oligonucleotide, an oligonucleotide duplex and combinations thereof may be imaged through hyperpolarized NMR by one or more of the following steps in any desirable order: (1) selective labeling of a nucleotide and/or nucleic acid molecule; (2) hyperpolarization of the nucleotide and/or nucleic acid molecule; (3) introduction of the hyperpolarized nucleotide and/or nucleic acid molecule into a cell whereby by the hyperpolarized nucleotide and/or nucleic acid molecule interacts with live genetic material within the cell; and (4) imaging of the live genetic material. The nucleic acid molecule may be an oligonucleotide, gene, DNA, RNA, an oligonucleotide duplex or combinations thereof. Hyperpolarization may be achieved through any known technique, including but not limited to PASADENA and DNP. Imaging may be achieved through any known technique, including NMR.

Notably, it is believed that only duplex DNA yield interpretable and reproducible ³¹P NMR spectra on hyperpolarization. DNP hyperpolaration is capable of distinguishing between single strand and double helix DNA in real time with enhanced signal. While 31P hyperpolarization probes the backbone dynamics and structural information; 15N and 13C hyperpolarization can provide information about the DNA base-pair stacking, dynamics and structure.

The present subject matter has disclosed novel methods and strategies accomplishing in vitro imaging of live genes. The use of PASADENA hyperpolarization techniques and DNP hyperpolarization techniques have provided a method of increasing MR signal to rival that of PET, without the negative effects, and have advanced strategies to image relevant live genes in vivo, and within the brain of a non-human primate.

The above disclosure generally describes the present subject matter. A more complete understanding can be obtained by reference to the following Examples, which are provided for purposes of illustration only and are not intended to limit the scope of the subject matter.

### EXAMPLES

The following examples describe a range of applications of the methods of the present subject matter, as well as a number of components that may be readily integrated and/or otherwise used in connection with the same. These Examples demonstrate some of the many steps of the methods of the subject matter, and the potential impact it may have on biological studies and the conventional practice of medicine. Modifications of these Examples will be readily apparent to those skilled in the art.

### Example 1

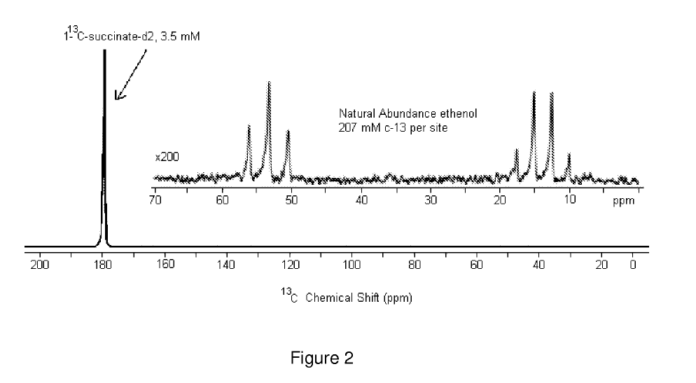

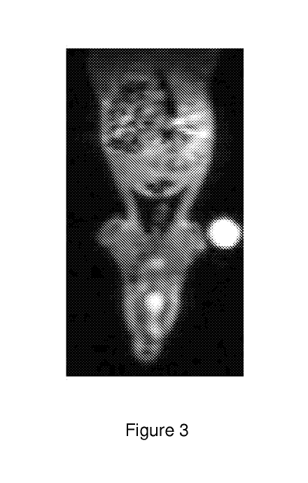

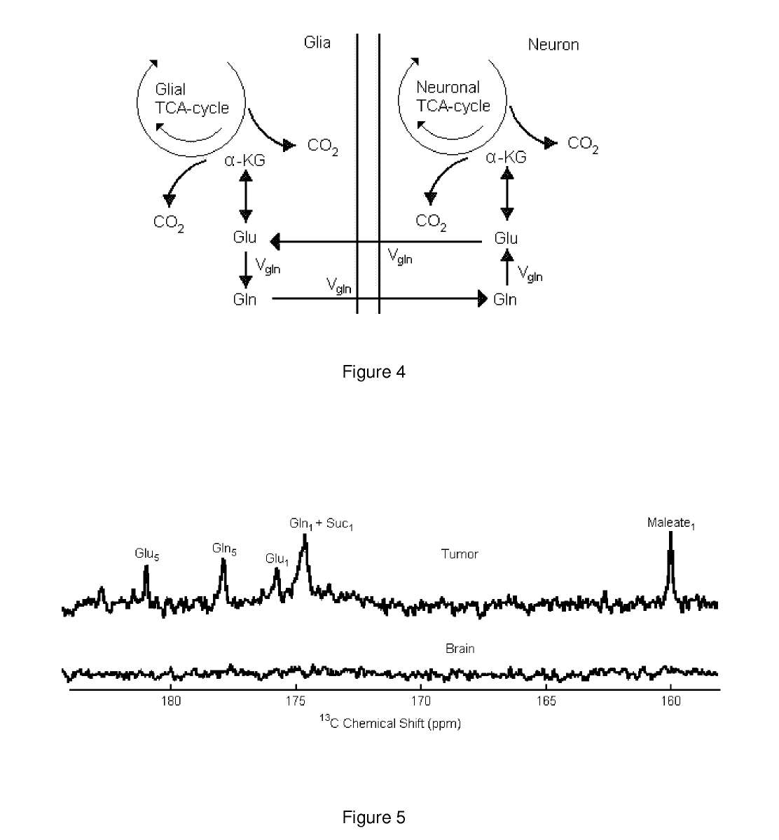

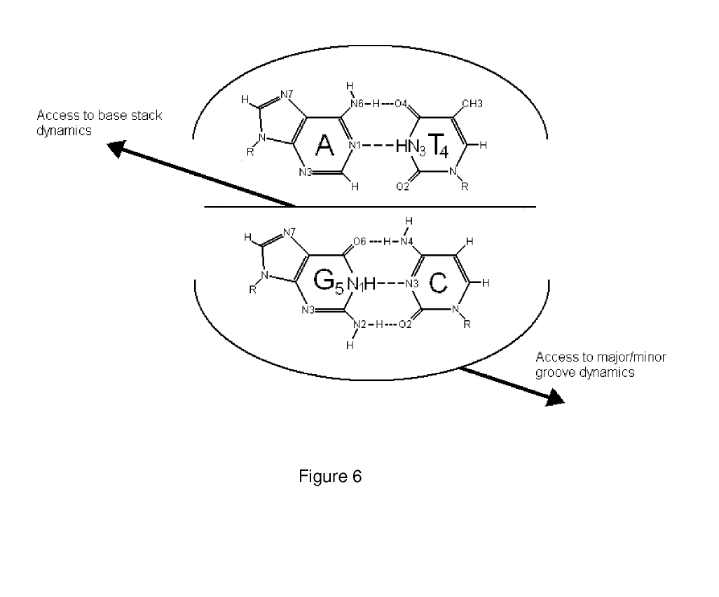

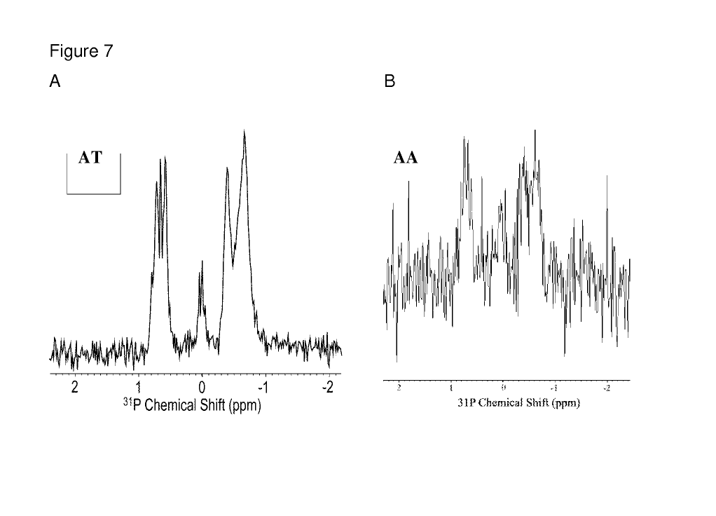

Cerebral imaging with hyperpolarized 13C succinate has been demonstrated following carotid artery injection in rats (FIG. 3) and consistently high levels of hyperpolarization have been achieved in vitro (FIG. 1 and FIG. 2). Using a 9L brain tumor model (Brian D Ross; U. of Michigan), differential uptake of maleate and/or succinate by tumor and exclusion of metabolites from normal brain was demonstrated (FIG. 4 and FIG. 5).

### Example 2

The best results come when hyperpolarized molecules are designed to answer a particular biological question, otherwise known as ‘hypothesis’ driven hyperpolarization. For example, mutant genes for human pancreatic cancers are commonly located in the short arm of chromosome One (Vogelstein, B., et al., DNA methylation and genetic instability in colorectal cancer cells. Proc Natl Acad Sci USA. March 18; 94(6): 2545-2550 [1997]). A novel pancreatic oncogene, succinic dehydrogenase (SDH) was mapped to the same chromosome, 1p. SDH triggered a cascade of abnormal metabolism. The inventors explored PASADENA imaging of this oncogene as a means of non-invasive early diagnosis of pancreatic cancer in humans.

13C-succinate supplied to a spheroid of living pancreatic cancer cells ‘MIAPACA’ was actively metabolized as demonstrated by 13C enriched products, glutamate, bicarbonate and citrate in the MR spectrum. When PASADENA precursor 13C fumaric acid was hyperpolarized with parahydrogen and the resulting hyperpolarized 13C succinate was injected in vivo, real-time in-vivo carbon-13 MR images and spectra of rat brain and abdomen were obtained. The images show hyperpolarized 1-¹³C-succinate to be a highly potent molecular imaging agent.

### Example 3

Live genes may be imaged through hyperpolarized NMR as follows. As an example, take a small primate, such as the Tupaia glis (or berlangi), the tree shrew, where in vivo neuroimaging with MRI and neurospectroscopy with proton MRS have proven merit in study of psychiatric disease (Van Der Hart, 2002). The inventors used Tupaia glis because of its close similarity in size to the laboratory rat where in vivo hyperpolarized MR images and spectra have already also been demonstrated (see above).

The next step is to establish a library of enriched (13C/¹⁵N) oligonucleotides, a series of target nucleotides, anti-sense-oligonucleotides and ‘small’ genes relevant to psychiatric disease, drug abuse and other brain disorders. Gene maps of amphetamine abuse and others have been created by others. Next, nucleotides should be hyperpolarized using one of the techniques discussed above. Strategic isotopic enrichment of oligonucleotides will give access to both the inside of the base-pair stack as well as the major/minor groove region of the DNA. Hyperpolarization of these nuclei will allow for probing both structural and dynamical elements of the gene.

Hyperpolarized nucleotides and oligonucleotides are introduced into the cell by conventional molecular biological techniques and the enriched cells are imaged with high resolution, low resolution and human MR scanners in real time. In vivo imaging can thus be accomplished in cell cultures, human white blood cells, and accessible organs, such as the liver and pancreas, as well as in tumor models.

We can now hyperpolarize individual nucleotides of DNA and RNA (A, T, G, C and U) at 13C and 15N nuclei. The 15N sites in oligonucleotides, DNA and genes located at the center of pi-stack of DNA helix and attached to exchangeable protons and thereby prone to drastic chemical shift changes monitored by NMR. It is well established that large oligonucleotides, DNA, RNA and genes can be selectively labeled by 13C/15N bases. The limitation of low 15N/13C SNR relevant to DNA, RNA and protein bimolecular NMR (currently NMR spectroscopy can be preformed on small DNA, RNA and proteins and that takes days to get interpretable multi-dimensional NMR data) can be largely removed by site selective isotope labeling of DNA followed by hyperpolarization. Thus, by hyperpolarization, we can directly probe the structure and dynamics inside DNA, RNA and a gene. The hyperpolarization technique may be extended to nucleotides, oligonucleotides, duplexes, DNA or RNA without prior substitution. Relying only upon the intrinsic 31 phosphorus content, ³¹P NMR signal is thereby enhanced to reveal, in a few seconds, the 31 phosphorus backbone of the oligonucleotide, DNA or gene. In the case that a single nucleotide mismatch (also known as single nucleotide polymorphism SNP) is present, this will be revealed by striking differences in the form of the 31P hyperpolarized spectrum. This technology can thereby be applied to imaging DNA, RNA, oligonucleotides, duplexes and SNP's and genes in seconds both in vivo and in vitro.

### Example 4

### Detection of Single Base-Pair Mismatches in Tumor Suppressor P51 Gene DNA Duplex by 31P Hyperpolarized NMR

### Background

DNA mismatches, or non-complementary base pairs, arise in vivo as a result of the misincorporation of bases during replication (Goodman, M. F., Creighton, S., Bloom, L. B., and Petruska, J. (1993) Biochemical basis of DNA-replication fidelity. Crit. Rev. Biochem. Mol. Biol., 28, 83-126), heteroduplex formation during homologous recombination (Bhattacharyya, A. and Lilley, D. M. (1989) Single base mismatches in DNA. Long- and short-range structure probed by analysis of axis trajectory and local chemical reactivity. J. Mol. Biol., 209, 583-597), mutagenic chemicals (Leonard, G. A., Booth, E. D. and Brown, T. (1990) Structural and thermodynamic studies on the adenine guanine mismatch in B-DNA. Nucleic Acids Res., 18, 5617-5623; Plum, G. E., Grollman, A. P., Johnson, F. and Breslauer, K. J. (1995) Influence of the oxidatively damaged adduct 8-oxodeoxyguanosine on the conformation, energetics, and thermodynamic stability of a DNA duplex. Biochemistry, 34, 16148-16160), ionizing radiation (Brown, T. (1995) Mismatches and mutagenic lesions in nucleic acids. Aldrichimica Acta, 28, 15-20) and spontaneous deamination (Lindahl, T. (1993) Instability and decay of the primary structure of DNA. Nature, 362, 709-715). These errors are usually detected and eliminated by DNA polymerase and a postreplicative mismatch repair system (Rajski, S. R., Jackson, B. A. and Barton, J. K. (2000) DNA repair: models for damage and mismatch recognition. Mutat. Res., 447, 49-72; Kolodner, R. (1996) Biochemistry and genetics of eukaryotic mismatch repair. Genes Dev., 10, 1433-1442; Modrich, P. (1991) Mechanisms and biological effects of mismatch repair. Annu. Rev. Genet., 25, 229-253). How these DNA mismatches are detected by the repair machinery of the cell requires an understanding at the molecular level. If not corrected, Single Nuclear Polymorphism (SNP) confers strong likelihood of diseases including inherited metabolic diseases, cancer and progressive senescence. Therefore, it has proved of considerable interest to characterize the structure, dynamics and biochemistry of mismatched base pairs in DNA and to determine how they affect the structure of the double helix in terms of both global and local perspectives.

The structures of several DNA duplexes containing mismatched base pairs have been characterized by X-ray crystallography (Brown, T., Kennard, S., Kneale, G. and Rabinovich, D. (1985) High resolution structure of a DNA helix containing mismatched base pairs. Nature, 315, 604-606; Hunter, W. N., Brown, T., Kneale, G., Anand, N. N., Rabinovich, D. and Kennard, O. (1987) The structure of guanosine-thymidine mismatches in B-DNA at 2.5 A Ê resolution. J. Biol. Chem., 262, 9962-9970; Hunter, W. N., Brown, T. and Kennard, O. (1987) Structural features and hydration of a dodecamer duplex containing two CA mispairs. Nucleic Acids Res., 15, 6589-6606) and NMR methods (Sowers, L. C., Fazakerley, G. V., Eritja, R., Kaplan, B. E. and Goodman, M. F. (1986) Base pairing and mutagenesis: observation of a protonated basepair between 2-aminopurine and cytosine in an oligonucleotide by proton NMR. Proc. Natl. Acad. Sci. USA, 83, 5434-5438; Fazakerley, G. V., Quignard, E., Woisard, A., Guschlbauer, W., Vandermarbel, G. A., Vanboom, J. H., Jones, M. and Radman, M. (1986) Structures of mismatched base pairs in DNA and their recognition by the

Escherichia coli mismatch repair system. EMBO J., 5, 3697-3703; Bhattacharya, P., Cha, J., and Barton, J. K. (2002) ¹H NMR determination of base-pair lifetimes in oligonucleotides containing single base mismatches. Nucleic Acids Res., 30, 4740-4750; Patel, D. J., Kozlowski, S. A., Ikuta, S, and Itakura, K. (1984) Deoxyadenosine pairing in the d(CGAGAATTCGCG) duplex conformation and dynamics at and adjacent to the dG.dA mismatch site. Biochemistry, 23, 3207-3217). In all of these structural studies, the mismatches were shown to have minimal effect on the global conformation of the DNA; the distortions produced are limited to the mismatched site and neighboring base pairs. ¹H NMR studies show that the mismatches GG (17-19), AA (20-22), TT (20-22), CC (23,24), GA (25-28) and GT (29) are well stacked in the helix and the bases remain in an intrahelix orientation. In fully base-paired right-handed B-form DNA duplexes, there are Nuclear Overhauser effects (NOEs) evident between base protons (H8 or H6) and the 5′-flanking sugar H1′ and H2′2″ protons, allowing an NOE “walk” from the 5′ to the 3′ end of the oligonucleotide. In mismatched duplexes, the NOE walk is conserved. This supports the notion that the mismatches are inserted and stacked well between the flanking base pairs, and that the oligonucleotides adopt the classical B form duplex, with minimal local disruption. ³¹P NMR studies also support a B1 conformation for DNA complexes which contain mismatched base pairs (Faibis, V., Cognet, J. A. H., Boulard, Y., Sowers, L. C. and Fazakerley, G. V. (1996) Solution structure of two mismatches GG and II in the K-ras gene context by nuclear magnetic resonance and molecular dynamics. Biochemistry, 35, 14452-14464; Cognet, J. A. H., Gabarro-Arpa, J., LeBret, M., Van der Marel, G. A., Boom, J. H. and Fazakerley, G. V. (1991) Solution conformation of an oligonucleotide containing a G.G mismatch determined by nuclear magnetic resonance and molecular mechanics. Nucleic Acids Res., 19, 6771-6779; Gervais, V., Cognet, J. A. H., LeBret, M., Sowers, L. C. and Fazakerley, G. V. (1995) Solution structure of two mismatches A_A and T_T in the K-ras gene context by nuclear magnetic resonance and molecular dynamics. Eur. J. Biochem., 228, 279-290; Boulard, Y., Cognet, J. A. H. and Fazakerley, G. V. (1997) Solution structure as a function of pH of two central mismatches, C′T and C′C, in the 29 to 39 K-ras gene sequence, by nuclear magnetic resonance and molecular dynamics. J. Mol. Biol., 268, 331-347).

Much less is known about the dynamic properties of the mismatched bases in the DNA duplex or how altered dynamics contribute to mismatch recognition. The bases in DNA move rapidly within the double helix, undergoing thermally driven structural fluctuations in solution. Since base motions occur within a multidimensional potential well determined by a combination of base-stacking and base-pairing forces, it is reasonable to expect that the motions of a mismatched base pair in DNA should be different from that of the fully matched pair; this dynamic difference may influence the interactions of mismatched base pairs with repair enzymes. Furthermore, the dynamics of mismatches, as well as fully matched base pairs, may play a pivotal role in modulating charge transport through DNA (Williams, T. T., Odom, D. T. and Barton, J. K. (2000) Variations in DNA charge transport with nucleotide composition and sequence. J. Am. Chem. Soc., 122, 9048-9049; Kelley, S. O., Boon, E. M., Barton, J. K., Jackson, N. M. and Hill, M. G. (1999) Single-base mismatch detection based on charge transduction through DNA. Nucleic Acids Res., 27, 4830-4837; Bhattacharya, P. K. and Barton, J. K. (2001) The influence of intervening mismatches on long-range guanine oxidation in DNA duplexes. J. Am. Chem. Soc., 123, 8649-8656).

The NMR studies described thus far in this Example were performed using hours to days of data acquisition on NMR spectrometers. The greatest limitation in all these earlier NMR studies was the lack of sensitivity. Because generally, NMR has been too insensitive, the dynamics of mismatched duplexes have been the focus of fluorescence-spectroscopy studies by Millar and co-workers (Guest, C. R., Hochstrasser, R. A., Sowers, L. C. and Millar, D. P. (1991) Dynamics of mismatched base pairs in DNA. Biochemistry, 30, 3271-3279). Time resolved fluorescence anisotropic decay measurements were obtained for a series of oligonucleotides containing intervening AP-X base pairs, where AP is the fluorescent adenine analog 2-aminopurine, and X=A, T, G or C. This technique allowed the detection of base motions in DNA on the picosecond timescale. Motions such as helical twisting, propeller twisting, base tilting and base rolling could potentially alter the emission dipole of AP, thereby contributing to changes in the decay of the fluorescence anisotropy. AP pairs differently with each of the different bases and these differences in its relative pairing ability were reflected in the internal dynamics. However, to achieve these results it was necessary to employ unnatural bases (AP), making it difficult to correlate with the “real” biochemical situation of natural DNA base sequences. A technique which can probe the internal dynamics of a DNA base sequence in real time without any unnatural substitutions would be of great value.

The main drawback of MR is its low sensitivity. Recent developments in Dynamic Nuclear Polarization (DNP) have enabled the MR signal of nuclei ¹³C and ¹⁵N to be increased by up to 4 orders of magnitude. This offers the potential to perform real-time imaging of precursors and their metabolic products shortly after introducing the hyperpolarized agent, with negligible background signal. Pyruvate is a substrate for several key metabolic processes that are implicated in cellular energy homeostasis and has been proposed as a new tool for metabolic imaging using hyperpolarized ¹³C. Recent advances have demonstrated the utility of this approach for detection of tumors in vivo, for diagnostic MR in a transgenic mouse model of prostate cancer, as well as for assessment of treatment response. The molecular targets of hyperpolarization have generally been previously enriched with the appropriate stable isotope. In this Example, the inventors extended the application of hyperpolarization to ³¹P and applied ³¹P NMR to detect single-base mismatch in short oligonucleotide duplexes. An advantage of ³¹P NMR, which has 100% natural abundance, is that no artificial isotope enrichment is needed, thereby making hyperpolarized ³¹P NMR of DNA duplexes a potentially versatile technique of detecting naturally occurring DNA mismatches.

Hyperpolarization, a method of enhancing NMR signal over 10,000 fold has not been previously applied to the question of DNA structure and dynamics. Here, the inventors conducted studies that establish the feasibility of employing hyperpolarized NMR for this purpose and illustrate the power of the technology for demonstrating the dynamics of single base mismatches within DNA duplexes. Using hyperpolarized ³¹P NMR, the inventors have defined sequences of fully matched base-pairs of varying length in a single scan NMR acquisition and to detect single base mismatches in a DNA duplex. Hyperpolarized ³¹P NMR spectroscopy offers a rapid and effective method of defining DNA structure and screening mismatches and SNPs, while the abnormalities detected in mismatched sequences underscore the importance of DNA sequence and of sequence context in governing base dynamics. Alternatively, hyperpolarization may usefully be applied to several other heteronuclei, including 13C and 15N which have been selectively enriched within DNA.

### Materials and Methods

### Oligonucleotide Preparation

Seven base-pair oligonucleotides were synthesized using standard phosphoramidite chemistry on an Applied Biosystems 392 DNA synthesizer with a dimethoxy trityl protecting group on the 5′ end (Caruthers, M. H., Barone, A. D., Beaucage, S. L., Dodds, D. R., Fisher, E. F., McBride, L. J., Matteucci, M., Stabinsky, Z. and Tang, J. Y. (1987) Chemical synthesis of deoxyoligonucleotides by the phosphoramidite

method. Methods Enzymol., 154, 287-313). Oligonucleotides were purified on a reversed phase Rainin Dynamax C18 column on a Waters HPLC and then deprotected by incubation in 80% acetic acid for 15 min. After deprotection, the oligonucleotides were purified again by HPLC. Following purification, these oligonucleotides were desalted on a Waters C18 SepPak column and then converted to a sodium salt using CM Sephadex C-25 (Sigma) equilibrated in NaCl. The concentration of the oligonucleotides was determined by UV-visible spectroscopy (Beckman DU 7400) using the extinction coefficients estimated for single-stranded DNA: ε(260 nm, M⁻¹ cm⁻¹) adenine (A)=15 400; guanine (G)=11 500; cytosine (C)=7400 and thymine (T)=8700. Single strands were mixed with equimolar amounts of complementary strand and annealed using a Perkin Elmer Cetus Thermal Cycler by gradual cooling from 90° C. to ambient temperature in 90 min. The concentrations of the samples varied between 50 and 100 μM duplex.

### Melting Temperature Experiments

To verify the presence of a mismatch in each of the sequences studied, the melting temperatures of the oligonucleotides were determined from absorbance versus temperature curves measured at 260 nm on a Beckman DU 7400 UV-visible spectrophotometer. Ten micromolar duplex was used in a buffer of 5 mM Na₂HPO₄, mM NaCl, pH 7.0. The melting profile of the duplexes was obtained by slowly lowering the temperature (0.5° C./min) from 75° C. to 10° C. and measuring the absorbance at 260 nm at each temperature. The Tm values represent the midpoint of the transition as obtained by fitting the melting profiles with a sigmoidal expression in Origin.

### Hyperpolarization and ³¹P NMR Spectroscopy

DNA duplexes were dissolved in an aqueous solution containing 15 mM of trityl free radical OX63 in H2O. The sample was polarized in a HyperSense DNP polarizer (Oxford Instruments Molecular Biotools, United Kingdom) at low temperature, 1.4 K, for 1 h in a superconducting magnet (3.35T) with microwave irradiation at 94 GHz. The polarized sample was dissolved in a 1 mL aqueous buffer solution prior to recording the spectrum in a 300 MHz Bruker Avance spectrometer.

### Results and Conclusions

### Sequence Design & Melting Temperature Studies

The general oligonucleotide sequence d(GACXGTC)₂; where X=G, T, A, C, was employed to determine the base-pair lifetimes for DNA duplexes containing different central single base mismatches (Table 2). A palindromic sequence was utilized that is self-complementary except at the mismatch site (X). Each of the sequences of these duplex is a portion of p51 genome sequence which comprises of these seven central palindromic base-pairs: 5′-GACAGTC-3′ (Osada, M., Ohba, M., Kawahara, C., Ishioka, C., Kanamaru, R., Katoh, I., Nimura, Y., Nakagawara, A., Obinata, M. and Ikawa, S. (1998) Cloning and functional analysis of human p51, which structurally and functionally resembles p53. Nat. Med., 4, 839-843). P51 gene is a tumor suppressor gene and its various polymorphisms are known to be associated with various forms of human cancer (Tani, M., Shimizu, K., Kawahara, C., Kohno, T., Ishimoto, O., Ikawa, S., and Yokota, J. (1999) Mutation and Expression of the p51 Gene in Human Lung Cancer. Neoplasia, 1, 71-79).

TABLE 2
Single base mismatch (X) Sequences of duplexes
used in DNP studies (Tm = Melting point)

| Duplexes* | Tm (° C.)** |
|---|---|
| Matched at X | |
| 5′-GACAGTC-3′ | 34.9 |
| 3′-CTGTCAC-5′ | |
| 5′-GACGGTC-3′ | 36.3 |
| 3′-CTGCCAC-5′ | |
| Mismatched at X | |
| 5′-GACAGTC-3′ | 28.1 |
| 3′-CTGACAC-5′ | |
| 5′-GACTGTC-3′ | 23.7 |
| 3′-CTGTCAC-5′ | |
| 5′-GACGGTC-3′ | 29.5 |
| 3′-CTGGCAC-5′ | |
| 5′-GACCGTC-3′ | 25.2 |
| 3′-CTGCCAC-5′ | |

*These sequences represent the six 7 mer oligonucleotides employed in 31P DNP hyperpolarization experiments.
**Melting temperatures (Tm) of the duplexes determined as described above. Samples include 10 mM duplex in a buffer of 5 mM Na₂HPO₄, 15 mM NaCl, pH 7.0.

Table 2 shows the melting temperatures for the two representative duplexes to demonstrate that duplexes containing mismatches are thermodynamically destabilized as compared with the matched pairs. For the mismatched duplexes, only one strand was used, resulting in a duplex with either AA, TT, CC or GG mismatch at the central site (X). To obtain a complementary base pair at site X, however, two different strands had to be used. This led to the possibility of the formation of a mixture of matched and mismatched duplexes as two separate strands were added to form the fully matched GC and AT sequences. Given the melting temperatures, that possibility was minimized by slow cooling during hybridization of the duplexes and repeated annealing of the sample to allow nucleation. The possibility of hairpin formation is negligible with these duplexes. Moreover, non-denaturing agarose gel electrophoresis with the mismatch-containing duplexes indicated no formation of hairpins in these duplexes (data not shown).

### Hyperpolarizing DNA Duplexes

Single scan hyperpolarized ³¹P NMR spectrum of a seven base-pair of fully-matched palindromic DNA duplex (5′-GACAGTC-3′) reveals seven distinct phosphorus resonances from the fourteen backbone phosphates because of the presence of C2 axis of symmetry due to self-complementary nature of the sequence (FIG. 7A). The phosphate resonances show the positions of three downfield AT and four upfield GC base-pairs and is consistent with previously reported ³¹P NMR of DNA duplex (Tani, M., Shimizu, K., Kawahara, C., Kohno, T., Ishimoto, O., Ikawa, S., and Yokota, J. (1999) Mutation and Expression of the p51 Gene in Human Lung Cancer. Neoplasia, 1, 71-79). The hyperpolarized ³¹P NMR of single strand do not reveal any ordered distinct resonances (not shown).

Single scan hyperpolarized ³¹P NMR spectra of a seven base-pair of single-MIS-matched (AA) palindromic DNA duplex (5′-GACAGTC-3′) reveals many more, well resolved phosphorus resonances than were seen in FIG. 7A signal (>7) from the fourteen backbone phosphates (FIG. 7B). The signal-to-noise is considerably reduced possibly due to increase backbone dynamics due to presence of central AA mismatch. Close inspection reveals upfield and downfield 31P resonances around 2 & −2 ppm. This can be readily explained by the reported structural and dynamical behavior of AA mismatches (Gervais, V., Cognet, J. A. H., LeBret, M., Sowers, L. C. and Fazakerley, G. V. (1995) Solution structure of two mismatches AA and TT in the K-ras gene context by nuclear magnetic resonance and molecular dynamics. Eur. J. Biochem., 228, 279-290; Maskos, K., Gunn, B. M., LeBlanc, D. A. and Morden, K. M. (1993) NMR study of G.A and A.A pairing in d(GCGAATAAGCG)₂. Biochemistry, 32, 3583-3595; Arnold, F. H., Wolk, S., Cruz, P. and Tinoco, I. J. (1987) Structure, dynamics, and thermodynamics of mismatched DNA oligonucleotide duplexes d(CCCAGGG)₂ and d(CCCTGGG)₂. Biochemistry, 26, 4068-4075; Boulard, Y., Cognet, J. A. H. and Fazakerley, G. V. (1997) Solution structure as a function of pH of two central mismatches, C′T and C′C, in the 29 to 39 K-ras gene sequence, by nuclear magnetic resonance and molecular dynamics. J. Mol. Biol., 268, 331-347; Peyret, P., Seneviratne, A., Allawi, H. T. and SantaLucia, J., Jr (1999) Nearest-neighbor thermodynamics and NMR of DNA sequences with internal AA, CC, GG, and TT mismatches. Biochemistry, 38, 3468-3477).

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

What is claimed is:

1. A method for imaging live genetic material, comprising:
providing a nucleotide and/or nucleic acid molecule selectively labeled with at least one stable isotope;
hyperpolarizing the nucleotide and/or nucleic acid molecule;
introducing the hyperpolarized nucleotide and/or nucleic acid molecule into a living cell whereby the hyperpolarized nucleotide and/or nucleic acid molecule interacts with live genetic material in the cell; and
imaging the live genetic material by magnetic resonance.

2. The method of claim 1, further comprising hyperpolarizing a number of intrinsic nuclei to reveal single nucleotide polymorphisms and/or point mutations in the live genetic material.

3. The method of claim 2, wherein the intrinsic nuclei comprise phosphorus of oligonucleotide and/or gene duplexes.

4. The method of claim 1, wherein providing the nucleotide and/or nucleic acid molecule comprises synthesizing the nucleotide and/or nucleic acid molecule.

5. The method of claim 1, wherein the nucleic acid molecule comprises a gene or segment thereof.

6. The method of claim 1, wherein the nucleic acid molecule comprises DNA or RNA.

7. The method of claim 1, wherein the nucleic acid molecule comprises an oligonucleotide.

8. The method of claim 1, wherein the nucleic acid molecule comprises a duplex.

9. The method of claim 1, wherein the at least one stable isotope is selected from the group consisting of carbon-13, nitrogen-15 and phosphorus-31.

10. The method of claim 1, wherein hyperpolarizing the nucleotide and/or nucleic acid molecule comprises Dynamic Nuclear Polarization (DNP) or Parahydrogen and Synthesis Allows Dramatically Enhanced Nuclear Alignment (PASADENA) hyperpolarization.

11. The method of claim 1, wherein imaging the live genetic material by magnetic resonance is performed in real time.

12. A method for studying the function of genetic material in vivo, comprising:
providing a nucleotide and/or nucleic acid molecule selectively labeled with at least one stable isotope;
hyperpolarizing the nucleotide and/or nucleic acid molecule;
introducing the hyperpolarized nucleotide and/or nucleic acid molecule into a living cell whereby the hyperpolarized nucleotide and/or nucleic acid molecule interacts with genetic material in vivo; and
imaging the genetic material by magnetic resonance to study its function.

13. The method of claim 12, further comprising hyperpolarizing a number of intrinsic nuclei to reveal single nucleotide polymorphisms and/or point mutations in the genetic material.

14. The method of claim 13, wherein the intrinsic nuclei comprise phosphorus of oligonucleotide and/or gene duplexes.

15. The method of claim 12, wherein providing the nucleotide and/or nucleic acid molecule comprises synthesizing the nucleotide and/or nucleic acid molecule.

16. The method of claim 12, wherein the nucleic acid molecule comprises a gene or segment thereof.

17. The method of claim 12, wherein the nucleic acid molecule comprises DNA or RNA.

18. The method of claim 12, wherein the nucleic acid molecule comprises an oligonucleotide.

19. The method of claim 11, wherein the nucleic acid molecule comprises a duplex.

20. The method of claim 12, wherein the at least one stable isotope is selected from the group consisting of carbon-13, nitrogen-15 and phosphorus-31.

21. The method of claim 12, wherein hyperpolarizing the nucleotide and/or nucleic acid molecule comprises Dynamic Nuclear Polarization (DNP) or Parahydrogen and Synthesis Allows Dramatically Enhanced Nuclear Alignment (PASADENA) hyperpolarization.

22. The method of claim 12, wherein imaging the genetic material by magnetic resonance is performed in real time.
